(19) United States
(12) Patent Application Publication
(10) Pub. No.: US 2026/0018255 A1
(43) Pub. Date: Jan. 15, 2026

(54) MULTI MODAL FLUID MEASUREMENT METHODS AND APPARATUS THEREFOR

(71) Applicant: TEF Technologies Inc., Burnaby (CA)

(72) Inventor: Babak Adeli KOUDEHI, Burnaby (CA)

(21) Appl. No.: 19/268,241

(22) Filed: Jul. 14, 2025

Related U.S. Application Data
(60) Provisional application No. 63/671,190, filed on Jul. 13, 2024.

(51) Int. Cl. G16C 20/20; G16C 20/30; G16C 20/70

(52) U.S. Cl. CPC G16C 20/20; G16C 20/30; G16C 20/70

(57) ABSTRACT

Training data is generated over a plurality of trials. In each trial, training information about a training fluid from sensors having at least two different sensing modalities is obtained. After generating training data, an artificial intelligence engine is trained on the training data. After training the artificial intelligence engine, the artificial intelligence engine infers a composition of a fluid based at least in part on deployed sensors. The artificial intelligence engine can be further trained using legacy sensors at an industrial facility.

### REFERENCE TO RELATED APPLICATIONS

This application claims priority from, and for the purposes of the United States of America the benefit under 35 USC 119 in relation to, U.S. Patent Application No. 63/671,190 filed 13 Jul. 2024, the entire disclosure of which is hereby incorporated herein by reference.

### TECHNICAL FIELD

The present technology relates to determining the composition of fluids. Example embodiments provide methods and systems for generating training data, training an artificial intelligence engine on such training data, and employing such artificial intelligence engines to infer a composition of a fluid based on outputs from multiple sensors having multiple corresponding sensor modalities.

### BACKGROUND

There is a general desire to ascertain the composition of fluids.

Fluid sensors are used in a large variety of industries including but not limited to industrial, agricultural, and domestic contexts.

Optical sensors and semiconductor sensors are two types of known sensors.

Optical sensors detect radiation (e.g., visible and/or UV light and/or infrared light) that has interacted with a fluid. Optical sensors may be sensitive over a particular range of wavelengths. Interaction between the optical sensor and incident radiation results in a change in electrical characteristics of the optical sensor (e.g., a current may be induced). Such electrical characteristics can be processed to infer one or more characteristics about the fluid.

Optical sensors have the advantage of being sensitive, selective, and having the ability to detect multiple constituents of a fluid simultaneously. Optical sensors are also non-invasive. Optical sensors are limited by environmental noise and narrow wavelength sensitivity.

An example type of semiconductor sensor is a photoelectrochemical sensor. Photoelectrochemical sensors comprise semiconductor material deposited over electrodes (or an alternative electrical measurement platform). Semiconductor materials are activated through exposure to photons with a minimum energy that is larger than the semiconductor band gap. In addition or alternatively, photoelectrochemical sensors can be activated by thermal energy, or electrical voltage. Physical or chemical interaction of fluid compounds with the semiconductor material results in a change of one or more electrical characteristics that are detectible at the electrodes.

Semiconductor sensors, of which photoelectrochemical sensors are a particular example, are sensitive. Relative to optical sensors, though, semiconductor sensors have the disadvantage of low selectivity and long-term stability.

The following references describe different types of sensors:
US20230014558A1
KR102510314B1
KR102656446B1
US20230358675A1
U.S. Pat. No. 10,101,266B2
WO2023075382A1
KR-10-2012-0030075

There remains a need for improved methods and apparatus for effectively sensing the composition of fluids.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

The following are some non-limiting example enumerated embodiments which illustrate various aspects of the invention:
1. A method for preparing a training data set for training an artificial intelligence engine (AIE) operable to infer a composition of a fluid, the method comprising:
   conducting a plurality of trials to generate a plurality of elements of the training data set, each trial comprising:
      obtaining a training fluid with a known composition;
      delivering the training fluid to a test chamber;
      obtaining first training information about the training fluid in the test chamber using one or more first training sensors having a first sensing modality;
      obtaining second training information about the training fluid in the test chamber using one or more second training sensors having a second sensing modality, the second sensing modality different from the first sensing modality; and
      associating, in a computer-interpretable format, the known composition with the first training information and the second training information to provide an element of the training data set.
2. The method as defined in aspect 1 (or any aspect herein) wherein the training fluid is a gas.
3. The method as defined in any one of aspects 1 to 2 (or any aspect herein) comprising varying a composition of the training fluid between successive trials.
4. The method as defined in any one of aspects 1 to 3 (or any aspect herein) wherein the first sensing modality comprises photoelectrochemical sensing, the one or more first training sensors comprise one or more photoelectrochemical sensors each comprising semiconductor material in electrical contact with one or more electrodes, each photoelectrochemical sensor operable to sense a change in electrical characteristics at the one or more electrodes when the semiconductor material interacts with the training fluid in the presence of incident radiation, and wherein the first training information is based on an output from the one or more photoelectrochemical sensors.
5. The method as defined in aspect 4 (or any aspect herein) wherein the one or more photoelectrochemical sensors comprise a plurality of photoelectrochemical sensors and the plurality of photoelectrochemical sensors comprises one or more of:
   each of the plurality of sensors comprising different crystalline structures;
   each of the plurality of sensors doped with different dopants;
   each of the plurality of sensors comprising different metallic nanoparticles comprising one or more of: Pt, Au, Ru, Pd, Ru, Rh, Ir, one or more oxides thereof, one or more complexes thereof, or a combination thereof; and
   each of the plurality of sensors fabricated using different synthesis techniques.
6. The method as defined in any one of aspects 4 to 5 (or any aspect herein) wherein the output from each of the one or more photoelectrochemical sensors comprises one or more photoelectrochemical response profiles, each photoelectrochemical response profile comprising a time dependent electrical characteristic measured at the one or more corresponding electrodes.
7. The method as defined in aspect 6 (or any aspect herein) wherein, for each trial, obtaining the first training information about the training fluid comprises one or more of:
   calculating a rate of change of the photoelectrochemical response profile and including the rate of change in the first training information; and
   calculating a maximum value of the photoelectrochemical response profile and including the rate of change in the first training information.
8. The method as defined in any one of aspects 1 to 7 (or any aspect herein) wherein the second sensing modality comprises optical sensing, the one or more second training sensors comprise one or more optical sensors, and wherein the second training information is based on an output of the one or more optical sensors.
9. The method as defined in aspect 8 (or any aspect herein) wherein the output of the one or more optical sensors comprises one or more electrical signals representative of an optical signature of the training fluid.
10. The method as defined in aspect 1 (or any aspect herein) wherein:
   the first sensing modality comprises photoelectrochemical sensing, the one or more first training sensors comprise one or more photoelectrochemical sensors each comprising semiconductor material in electrical contact with one or more electrodes, each photoelectrochemical sensor operable to sense a change in electrical characteristics at the one or more electrodes when the semiconductor material interacts with the training fluid in the presence of incident radiation, and wherein the first training information is based on an output from the one or more photoelectrochemical sensors;
   the second sensing modality comprises optical sensing, the one or more second training sensors comprise optical sensors, and wherein the second training information is based on an output of the one or more optical sensors;
   wherein the method comprises, for each trial:
      emitting radiation from at least one radiation emitter, the radiation directed to impinge on the one or more first training sensors and the one or more second training sensors; and
      associating, in the computer-interpretable format, an emission profile of the at least one radiation emitter with the element of the training data set.
11. The method as defined in aspect 10 (or any aspect herein) wherein the at least one radiation emitter comprises a plurality of radiation emitters, wherein the method comprises emitting different wavelengths of radiation from the plurality of radiation emitters.
12. The method as defined in any one of aspects 10 to 11 (or any aspect herein) comprising varying the wavelengths of radiation emitted from the at least one radiation emitter between successive trials.
13. The method as defined in any one of aspects 1 to 12 (or any aspect herein) comprising:
   for each trial, one or both of:
      obtaining a humidity measurement of the training fluid, and associating, in the computer-interpretable format, the humidity measurement with the element of the training data set; and
      obtaining a temperature measurement of the training fluid, and associating, in the computer-interpretable format, the temperature measurement with the element of the training data set.
14. A method for training an artificial intelligence engine (AIE) operable to infer a composition of a fluid, the method comprising:
   (a) employing the method of any one of aspects 1 to 13 (or any aspect herein) to generate a training data set;
   (b) providing an AIE comprising trainable parameters;
   (c) initializing values for the trainable parameters;
   (d) performing a plurality of training iterations to obtain a trained AIE comprising trained parameters, each training iteration comprising, for an element of the training data set:
      (i) predicting a composition of the training fluid based at least in part on the first training information, the second training information, and current values of the trainable parameters;
      (ii) determining an error between the predicted composition of the training fluid from step (i) and the known composition of the training fluid; and
      (iii) modifying, based at least in part on the error calculated in step (ii), the current values of the trainable parameters to obtain updated parameters.
15. The method as defined in aspect 14 (or any aspect herein) wherein each training iteration comprises evaluating one or more training conclusion conditions, and, if the one or more training conclusion conditions are met, ceasing the training iterations to thereby obtain the trained AIE, the training conclusion conditions comprising one or more of:
   the error between the predicted composition and the known composition dropping below a configurable error threshold for a configurable number of training iterations;
   the error between the predicted composition and the known composition not reducing by less than a configurable reduction threshold for a configurable number of reduction iterations; and
   a number of training iterations exceeding a configurable threshold number of training iterations.
16. The method as defined in any one of aspects 14 to 15 (or any aspect herein) wherein the element of the training data set comprises additional data comprising one or more of:
   an emission profile;
   a relative humidity measurement of the training fluid; and
   a temperature measurement of the training fluid; and
   step (i) comprises predicting a composition of the training fluid for the at least one element of the training data set based at least in part on the first training information, the second training information, the current values of the trainable parameters, and the additional data.
17. A method of inferring a composition of a fluid, the method comprising: providing one or more first deployed sensors having the first sensing modality, and one or more second deployed sensors having the second modality;
   acquiring the trained AIE from any one of aspects 14 to 16 (or any aspect herein);
   obtaining first information about the fluid from the one or more first deployed sensors;
   obtaining second information about the fluid from the one or more second deployed sensors; and
   inferring, using the trained AIE, an inferred composition of the fluid based at least in part on the first information, the second information, and the trained parameters of the trained AIE.
18. The method as defined in aspect 17 (or any aspect herein) comprising:
   obtaining additional deployed data about the fluid, the additional deployed data comprising one or more of:
      a deployed emission profile of one or more deployed radiation emitters emitting radiation directed to impinge on the fluid;
      a deployed temperature measurement of the fluid; and
      a deployed relative humidity measurement of the fluid; and
   inferring, using the AIE, the inferred composition of the fluid based at least in part on the first information, the second information, the trained parameters of the AIE, and the additional deployed data.
19. The method as defined in any one of aspects 17 to 18 (or any aspect herein) wherein:
   the first deployed sensors comprise the first training sensors; and
   the second deployed sensors comprise the second training sensors.
20. The method as defined in any one of aspects 17 to 19 (or any aspect herein) comprising:
   obtaining a reference composition of the fluid;
   determining a reference error between the inferred composition of the fluid and the reference composition of the fluid; and
   modifying the values of the trained parameters based on the reference error.
21. The method as defined in aspect 20 (or any aspect herein) comprising obtaining the reference composition from one or more of:
   a legacy sensor operable to determine the reference composition; and
   historical expected values for the composition of the fluid.
22. A method for training an artificial intelligence engine (AIE) operable to infer a composition of a fluid, the method comprising:
   conducting a plurality of trials to generate a plurality of elements of a training data set, each trial comprising:
      obtaining a training fluid with a known composition;
      delivering the training fluid to a test chamber;
      obtaining first training information about the training fluid in the test chamber using one or more first training sensors having a first sensing modality;
      obtaining second training information about the training fluid in the test chamber using one or more second training sensors having a second sensing modality, the second sensing modality different from the first sensing modality;
      associating, in a computer-interpretable format, the known composition with the first training information and the second training information to provide an element of the training data set; and
   providing an AIE comprising trainable parameters;
   initializing values for the trainable parameters;
   performing a plurality of training iterations to obtain a trained AIE comprising trained parameters, each training iteration comprising, for an element of the training data set:
      (i) predicting a composition of the training fluid based at least in part on the first training information, the second training information, and current values of the trainable parameters;
      (ii) determining an error between the predicted composition of the training fluid from step (i) and the known composition of the training fluid; and
      (iii) modifying, based at least in part on the error calculated in step (ii), the current values of the trainable parameters to obtain updated parameters.
23. The method as defined in aspect 22 (or any other aspect herein) comprising:
   for each trial:
      emitting radiation from at least one radiation emitter, the radiation directed to impinge on the one or more first training sensors and the one or more second training sensors; and
      associating, in the computer-interpretable format, an emission profile of the at least one radiation emitter with the element of the training data set; and
   for each training iteration:
      predicting a composition of the training fluid for the at least one element in the training data set based at least in part on the first training information, the second training information, the emission profile, and current values of the trainable parameters.
24. The method as defined in any one of aspects 22 to 23 (or any other aspect herein) comprising any of the features, combinations of features and/or sub-combinations of features of any of aspects 1 to 21.
25. A method of inferring a composition of a fluid, the method comprising:
   conducting a plurality of trials to generate a plurality of elements of a training data set, each trial comprising:
      obtaining a training fluid with a known composition;
      delivering the training fluid to a test chamber;
      obtaining first training information about the training fluid in the test chamber using one or more first training sensors having a first sensing modality;
      obtaining second training information about the training fluid in the test chamber using one or more second training sensors having a second sensing modality, the second sensing modality different from the first sensing modality;
      associating, in a computer-interpretable format, the known composition with the first training information and the second training information to provide an element of the training data set;
   providing an AIE comprising trainable parameters;
   initializing values for the trainable parameters;
   performing a plurality of training iterations to obtain a trained AIE comprising trained parameters, each training iteration comprising, for an element of the training data set:
      (i) predicting a composition of the training fluid based at least in part on the first training information, the second training information, and current values of the trainable parameters;
      (ii) determining an error between the predicted composition of the training fluid from step (i) and the known composition of the training fluid; and
      (iii) modifying, based at least in part on the error calculated in step (ii), the current values of the trainable parameters to obtain updated parameters;
   providing one or more first deployed sensors having the first sensing modality, and one or more second deployed sensors having the second modality;
   obtaining first information about the fluid from the one or more first deployed sensors;
   obtaining second information about the fluid from the one or more second deployed sensors; and
   inferring, using the trained AIE, an inferred composition of the fluid based at least in part on the first information, the second information, and the trained parameters of the trained AIE.
26. The method as defined in aspect 25 (or any aspect herein) wherein:
   the first deployed sensors comprise the first training sensors; and
   the second deployed sensors comprise the second training sensors.
27. The method as defined in any one of aspects 25 to 26 (or any other aspect herein) comprising any of the features, combinations of features and/or sub-combinations of features of any one of aspects 1 to 21.
28. An apparatus for determining the composition of fluids, the apparatus comprising: one or more first sensors having a first sensing modality;
   one or more second sensors having a second sensing modality; and
   a processor configured to:
      obtain first information about an unknown fluid from the one or more first sensors;
      obtain second information about the unknown fluid from the one or more second sensors; and
      infer, using a trained AIE comprising trained parameters, an inferred composition of the unknown fluid based at least in part on the first information, the second information, and the trained parameters of the trained AIE.
29. The apparatus of aspect 28 (or any aspect herein) wherein:
   the one or more first sensors comprise one or more photoelectrochemical sensors each comprising semiconductor material in electrical contact with one or more electrodes, each photoelectrochemical sensor operable to sense a change in electrical characteristics at the one or more electrodes when the semiconductor material interacts with the unknown fluid in the presence of incident radiation, and wherein the first information is based on an output from the one or more photoelectrochemical sensors; and
   the one or more second sensors comprise one or more optical sensors, and wherein the second information is based on an output of the one or more optical sensors.
30. The apparatus of any one of aspects 28 to 29 (or any aspect herein) wherein the processor is operable to generate a training data set in response to receipt of training data, wherein generating the training data set comprises generating a plurality of elements of the training data set over a plurality of trials, each trial comprising:
   obtaining a training fluid with a known composition;
   delivering the training fluid to a test chamber;
   obtaining, using the processor, first training information about the training fluid in the test chamber using the one or more first sensors having the first sensing modality;
   obtaining, using the processor, second training information about the training fluid in the test chamber using the one or more second sensors having the second sensing modality; and
   associating in a computer-interpretable format, using the processor, the known composition with the first training information and the second training information to provide an element of the training data set.
31. The apparatus of aspect 30 (or any aspect herein) wherein the processor is operable to train, using the training data, an untrained AIE comprising trainable parameters to obtain the trained AIE comprising trained parameters, wherein the training comprises:
   initializing values for the trainable parameters; and
   performing a plurality of training iterations to obtain the trained AIE comprising trained parameters, each training iteration comprising, for an element of the training data set:
      (i) predicting a composition of the training fluid based at least in part on the first training information, the second training information, and current values of the trainable parameters;
      (ii) determining an error between the predicted composition of the training fluid from step (i) and the known composition of the training fluid; and
      (iii) modifying, based at least in part on the error calculated in step (ii), the current values of the trainable parameters to obtain updated parameters.
32. The apparatus of any one of aspects 28 to 31 (or any aspect herein) comprising (e.g., by suitable configuration of the processor) any of the features, combinations of features, and/or sub-combinations of features of any one of aspects 1 to 21.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a flow chart of a method of inferring a composition of a fluid according to an example embodiment.

FIG. 2A is a flow chart of a method for generating the FIG. 1 training data set according to an example embodiment.

FIG. 2B is a schematic of a training data element according to an example embodiment.

FIG. 3 is a graph showing photoelectrochemical responses as a function of time for a gas comprising NO₂ at varying concentrations.

FIG. 4A is a graph showing photoelectrochemical responses as a function of time to gases with different concentrations of HCHO. FIG. 4B is a graph showing the relationship between the average maximum absolute value of the FIG. 4A responses and HCHO concentrations. FIG. 4C is a graph showing the relationship between the absolute value of the response curve slope in FIG. 4A 5 minutes after being exposed to HCHO, and HCHO concentration.

FIG. 5A is a graph showing absorbance as a function of wavenumber for a number of exemplary gases.

FIG. 5B is a graph showing absorbance for two gases as a function of wavelength, and photosensitivity of an example optical sensor as a function of wavelength according to an example embodiment.

FIG. 6A is a diagram of an apparatus for generating training data according to an example embodiment.

FIG. 6B is a radiation emitter according to an example embodiment.

FIG. 6C is a photoelectrochemical sensor according to an example embodiment.

FIG. 6D is an optical sensor according to an example embodiment.

FIG. 6E is a diagram of a sensor array that can be used in the FIG. 6A apparatus according to an example embodiment.

FIG. 6F is a diagram of a radiation emitter array that can be used in the FIG. 6A apparatus according to an example embodiment.

FIG. 7 is a flow chart of a method for training an AIE comprising a plurality of trainable parameters according to an example embodiment.

FIG. 8A is a flow chart of a method for inferring a composition of a fluid according to an example embodiment.

FIG. 8B is a diagram of an apparatus for inferring a composition of a fluid according to an example embodiment.

FIG. 9 is a flow chart of a method for training a deployed sensor using a reference measurement according to an example embodiment.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Aspects of the invention provide methods and apparatus for predicting the composition of fluids which leverage synergies from the combined use of more than one sensing modality together with artificial intelligence. One aspect of the present technology provides methods for generating labelled training data in the form of known fluid compositions and training information from different sensors having different sensing modalities. The training data can be used to train the parameters of an artificial intelligence engine (AIE). The trained AIE can be used to infer a composition of a fluid based on an output from the multiple sensing modalities. A trained AIE which uses information from at least two different sensing modalities may infer the compositions of fluids.

The term “composition” when used in reference to a fluid herein refers to the identity of one or more constituents of that fluid, and optionally also refers to the concentration of one or more of the identified constituents.

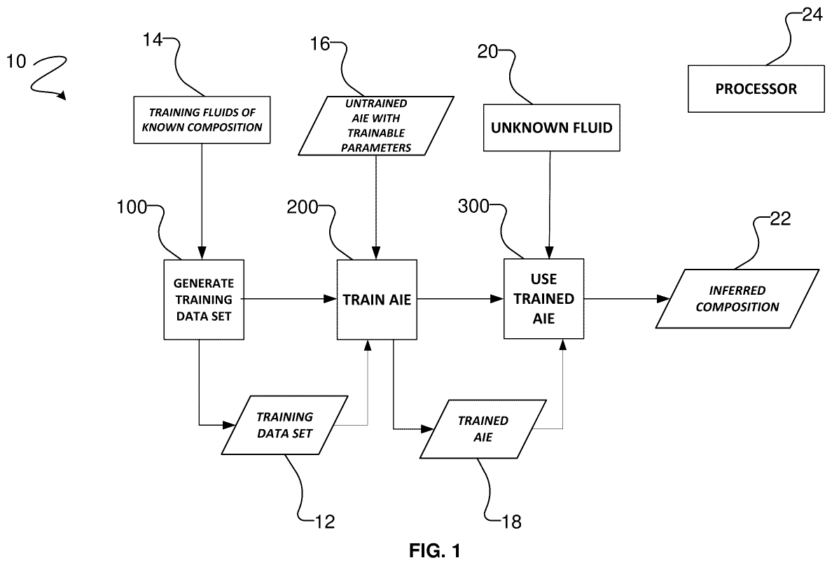

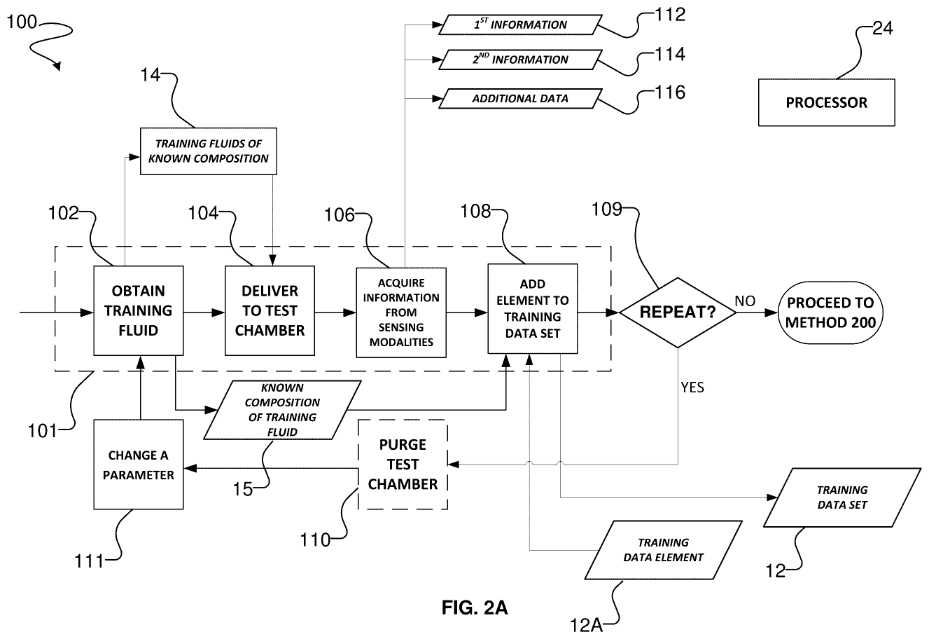

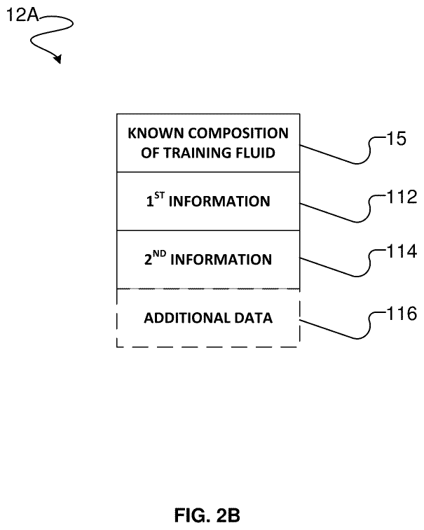

FIG. 1 is a flowchart of a method 10 for inferring a composition of an unknown fluid 20 according to an example embodiment. Method 100 may be performed by a suitably configured (e.g., programmed) processor 24 which may be part of a suitably configured computer system (not shown). Method 10 commences in step 100 which comprises generating a training data set 12 based on a number of training fluids 14 with a known composition 15. Each element of training data set 12 may comprise, for example, a known composition 15 of a training fluid 14, along with training information (e.g., first information 112, second information 114, and additional data 116—see FIG. 2) about the training fluid 14 obtained from sensors (not shown) having different sensor modalities. Step 200 receives, as input, training data 12 generated in step 100 and an untrained artificial intelligence engine (AIE) 16 comprising a number of trainable parameters (not expressly shown). Step 200 comprises training untrained AIE 16 using training data 12 to thereby obtain trained AIE 18. Step 300 comprises utilizing trained AIE 18 to infer the composition (inferred composition 22) of a previously unknown fluid 20.

FIG. 2A is a flow chart of a method 100 of generating a training data set 12 according to an example embodiment. Method 100 may be performed by processor 24 which may be embodied by the same processor(s) used for other methods described herein and/or by different processor(s).

Method 100 comprises conducting a plurality of trials 101. Completion of each trial 101 results in the addition of an element 12A to training data set 12.

Step 102 comprises obtaining a training fluid 14. In some embodiments, training fluid 14 is a gas. In some embodiments, training fluid 14 is a mixture of one or more gases. By way of non-limiting example, the one or more gases could be one or more of: air (e.g., a mixture of approximately 78% nitrogen, 21% oxygen, and 1% argon), nitrogen, carbon dioxide, methane, one or more sulfur oxides (e.g., sulfur dioxide and/or sulfur trioxide), one or more nitrogen oxides (e.g., nitric oxide, and/or nitrogen dioxide), carbon monoxide, ammonia, ozone, oxygen, hydrogen, ethanol, methanol, formaldehyde, acetylene, propane, butane, chlorine, chlorine dioxide, bromine, hydrogen sulfide, hydrogen fluoride, hydrogen chloride, phosphine, arsine, phosgene, and/or one or more volatile organic compounds (VOCs).

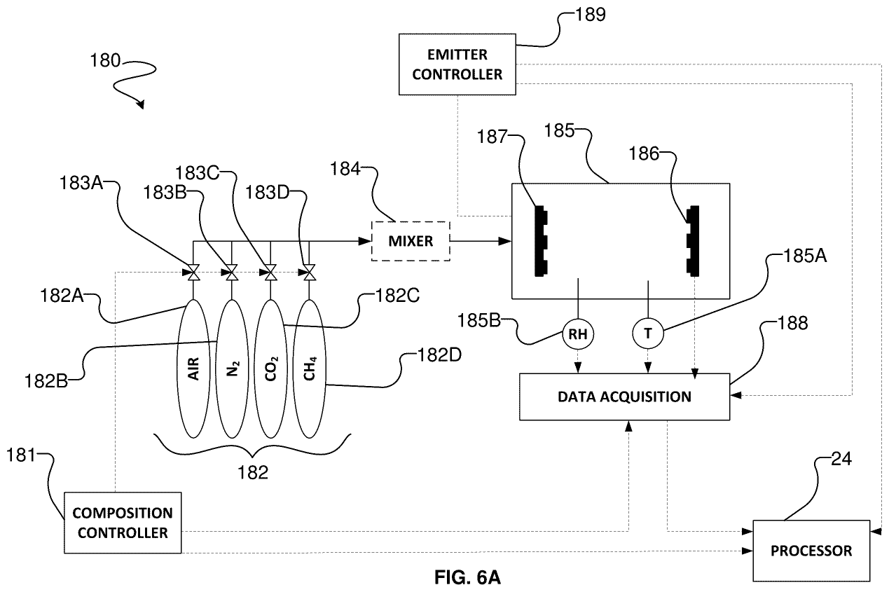

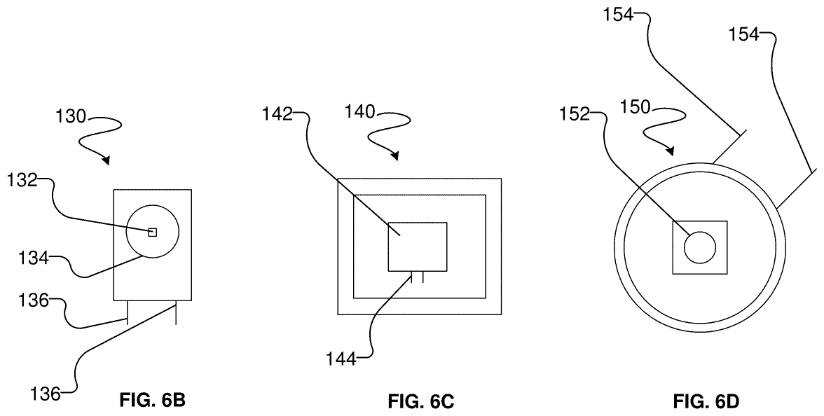

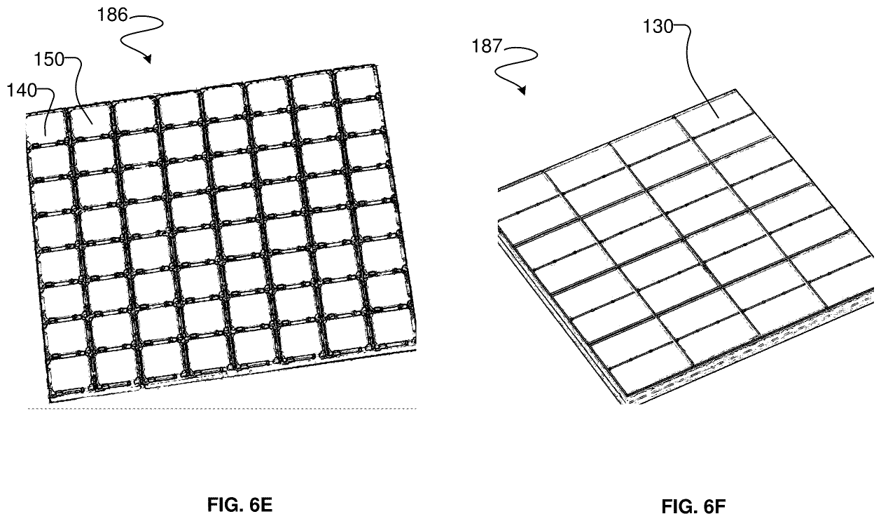

Step 104 comprises delivering training fluid 14 to a test chamber (e.g., test chamber 185 in FIG. 6A) with a plurality of sensors sensitive to training fluid 14 in the test chamber. In some embodiments, the test chamber comprises a plurality of sensors on a sensing array (e.g., sensor array 186 in FIG. 6A).

In some embodiments, the plurality of sensors comprise at least two different sensing modalities. In some embodiments, the at least two different sensing modalities comprise optical sensing, and photoelectrochemical sensing. In such embodiments, the plurality of sensors include one or more optical sensors and one or more photoelectrochemical sensors. Since the sensors (e.g., sensors on sensor array 186 in FIG. 6A) are used to generate training data, they can be referred to as training sensors.

In some embodiments, training fluid 14 in the test chamber is exposed to radiation to facilitate the acquisition of information about training fluid 14 from the training sensors.

FIG. 6B is a radiation emitter 130 according to an example embodiment. In the FIG. 6B embodiment, radiation emitter 130 is a light emitting diode (LED). Radiation emitter 130 comprises die 132 which is operable to emit radiation. Die 132 comprises a PN junction. Die 132 is situated on substrate 134. Leads 136 may couple to a source of electrical energy and may be configured to provide a current to die 132 to thereby cause die 132 to emit radiation.

In some embodiments, one or more radiation emitters 130 are directed to impinge on training fluid 14 in the test chamber. For example, the one or more radiation emitters 130 may be situated on a radiation emitter array within the test chamber (e.g., radiation emitter array 187 in test chamber 185). By way of further example, the one or more radiation emitters may be configured (e.g., using suitable optical elements such as lenses, mirrors, waveguides, fiber optics, or the like) to impinge on training fluid 14 in the test chamber.

In some embodiments, the one or more radiation emitters 130 emit a spectrum of radiation. For example, the one or more radiation emitters 130 may emit radiation across a spectrum ranging between deep ultraviolet to infrared. In some embodiments, this spectrum is more narrow. In some embodiments, the one or more radiation emitters 130 comprise a plurality of radiation emitters 130 that emit radiation in spectra that overlap.

Returning to FIG. 2A, step 106 comprises obtaining training information (e.g. first information 112, second information 114) from the at least two different sensing modalities.

As mentioned above, in some embodiments the training sensors comprise one or more photoelectrochemical sensors. FIG. 6C is a photoelectrochemical sensor 140 according to an example embodiment. Photoelectrochemical sensor 140 comprises sensing material 142 that is operable to interact with analyte (e.g., training fluid 14). Sensing material 142 is electrically coupled to electrodes 144. In some embodiments, sensing material 142 may be situated atop electrodes 144 that may be interdigitated to increase the sensitivity of photoelectrochemical sensor 140. Interaction of sensing material 142 with an analyte results in a change in one or more electrical characteristics of sensing material 142 that are detectible by the electrodes 144.

When the training sensors comprise one or more photoelectrochemical sensors 140, the training information (e.g., first information 112) from photoelectrochemical sensors 140 comprises one or more photoelectrochemical response profiles.

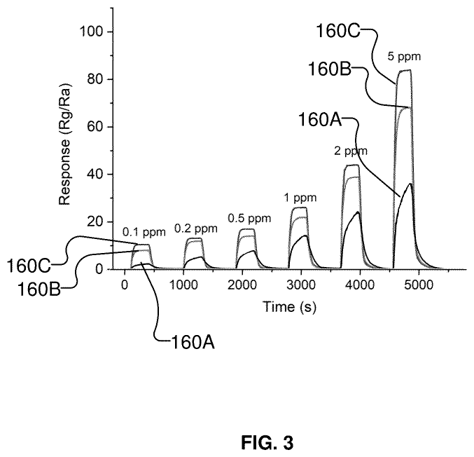

FIG. 3 shows photoelectrochemical sensor response profiles as a function of time for a gas comprising NO₂ at progressively increasing concentrations. In the FIG. 3 example, the y axis represents the ratio between resistance measured by the electrodes in the photoelectrochemical sensor relative to the resistance measured by the electrodes in the presence of a reference gas.

The temporally overlapping photoelectrochemical response profiles in FIG. 3 correspond to photoelectrochemical sensors that are made using different synthesis techniques and/or that are doped differently and/or that are modified physically or chemically differently.

Response profile 160A corresponds to a tungsten-oxide based photoelectrochemical sensor prepared using physical vapour deposition (PVD) with zero angle between the normal vector on the substrate surface and the direction of evaporation. Response profile 160B corresponds to a tungsten-oxide based photoelectrochemical sensor prepared using PVD with 75 degree angle between the normal vector on the substrate surface and the direction of evaporation. Response profile 160C corresponds to a tungsten-oxide based photoelectrochemical sensor prepared using PVD with 75 degree angle between the normal vector on the substrate surface and the direction of evaporation and doped with gold.

Tungsten oxide-based photoelectrochemical sensors are just one example of photoelectrochemical sensors that could be used. In some embodiments, photoelectrochemical sensors comprising ZnO, Ga₂O₃, GaN, SnO₂, TiO₂, Fe₂O₃, In₂O₃, InP, GaAs, Si, SiC, Ge, MoS₂, Graphene, ZrO₂, GeO₂ and/or any combination thereof could be used.

As shown in FIG. 3, different photoelectrochemical sensors (e.g., photoelectrochemical sensors synthesized and/or fabricated using different synthesis/fabrication techniques and/or photoelectrochemical sensors doped with different dopants) exhibit different response profiles to the same analyte. FIG. 3 also shows that the maximum value of the response correlates with the concentration of analyte. For example, at 0.1 ppm of NO₂, the maximum value of the photoelectrochemical response for each of profiles 160A, 160B, and 160C is small in comparison to the maximum value of the respective response profiles 160A, 160B, 160C at 5 ppm.

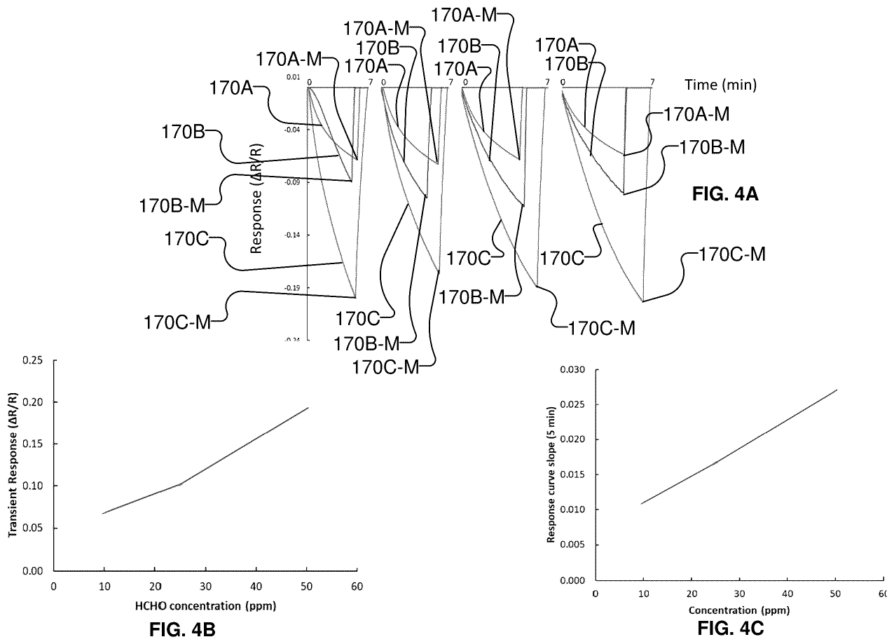

Another example set of photoelectrochemical response profiles is shown in FIG. 4A, which shows a series of photoelectrochemical response profiles as a function of time for different concentrations of formaldehyde (HCHO). Profiles 170A correspond to 10 ppm of HCHO, profiles 170B correspond to 25 ppm of HCHO, and profiles 170C correspond to 50 ppm of HCHO.

In FIG. 4A, the x axes represent time (in minutes), and the y axis represents the relative difference of the photoelectrochemical sensor resistance relative to a reference resistance of the photoelectrochemical sensor in a clean air reference.

FIG. 4B shows the average absolute value of the extremum values of the FIG. 4A responses (i.e., the absolute value of the average of the extrema shown by callouts 170A-M, 170B-M, and 170C-M in FIG. 4A).

FIG. 4C shows the average slope of the FIG. 4A photoelectrochemical response curves 5 minutes after being exposed to HCHO.

As shown in FIG. 4B, the absolute value of the extrema values of the photoelectrochemical response profiles increase approximately linearly with increasing HCHO concentration. As shown in FIG. 4C, the slope of the FIG. 4A response profiles increase approximately linearly with increasing HCHO concentration.

FIG. 3 shows that different photoelectrochemical sensors (e.g., photoelectrochemical sensors made using different synthesis and/or fabrication techniques and/or photoelectrochemical sensors that are doped differently) can have different photoelectrochemical response profiles to the same analyte. FIG. 3, FIG. 4A, FIG. 4B, and FIG. 4C show that the same photoelectrochemical sensor can have a different photoelectrochemical response profile in response to the same analyte at different concentrations.

Without wishing to be bound by theory, the inventor posits that photoelectrochemical sensors with different crystalline structures and/or photoelectrochemical sensors having one or more metallic particles deposited thereon (e.g., Pt, Au, Ru, Pd, Ru, Rh, Ir, one or more oxides thereof, or one or more complexes thereof) and/or photoelectrochemical sensors with different chemical compositions also exhibit different response profiles to the same analyte. The inventor further posits that photoelectrochemical response profiles are dependent on the emission profile of radiation incident on the photoelectrochemical sensor from the radiation emitters.

The FIG. 3 and FIG. 4A photoelectrochemical response profiles are examples. In some embodiments, photoelectrochemical response profiles as described herein could comprise any electrical characteristic detectible in connection with one or more electrodes in a photoelectrochemical sensor.

As mentioned above, in some embodiments the training sensors also comprise one or more optical sensors. In such embodiments, the training information (e.g., second information 114) from the optical sensors comprises one or more optical signatures of the training fluid.

FIG. 6D is an optical sensor 150 according to an example embodiment. Optical sensor 150 comprises sensing material 152. Incident radiation on sensing material 152 may change the electrical properties of the sensing material 152, which is detectible by one or more electrodes 154. Sensing material 152 may be sensitive to incident radiation at a particular wavelength or range of wavelengths.

The output of an optical sensor 150 may take the form of an electrical characteristic (e.g., a current, voltage, and/or the like) or a change in an electrical characteristic, which may be referred to as an optical signature.

In some embodiments, second information 114 may comprise one or more optical signatures from one or more optical sensors 150.

The optical signature detected by optical sensor 150 may depend on the wavelength of radiation incident on the optical sensor 150, the composition of the optical sensor 150, a temperature of the medium, a relative humidity of the medium, and the like.

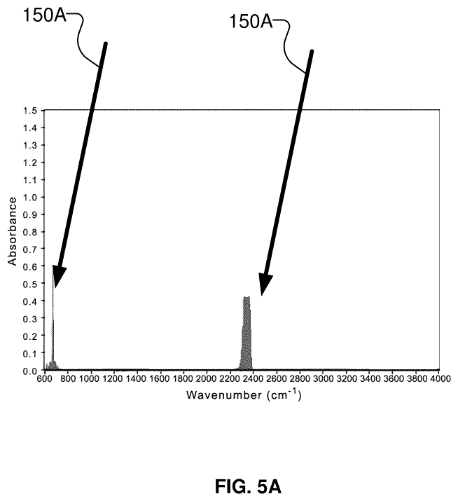

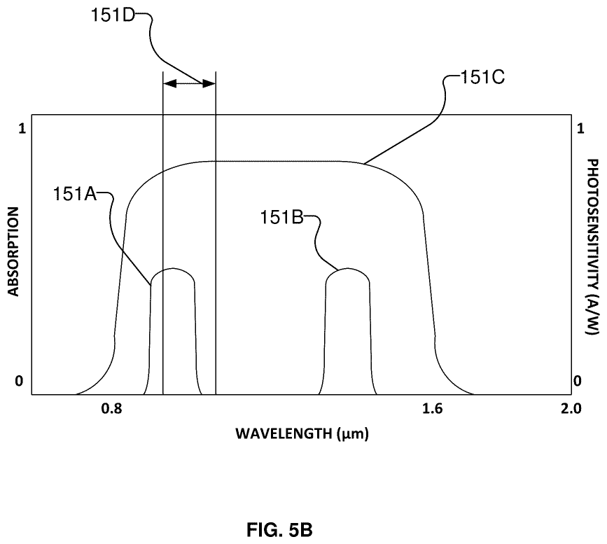

The optical signature detected by optical sensor 150 may also depend on the presence or absence of a particular analyte (e.g., the presence or absence of one or more constituents of unknown fluid 20). FIG. 5A shows absorbance as a function of wavenumber (λ⁻¹) for a number of exemplary gases.

Different gases absorb photons at different wavelengths. Consequently, different gases have different optical absorbance profiles.

For example, as shown in FIG. 5A, a gas may have an optical absorbance profile that exhibits two peaks at wavenumbers of approximately 650 cm⁻¹ and 2300 cm⁻¹, as shown by arrows 150A. In such a case, an optical sensor sensitive to radiation with a wavenumber of 2300 cm⁻¹ might exhibit a change in current in the presence of such a gas, whereas an optical sensor sensitive to radiation with a wavenumber of 3000 cm⁻¹ might not exhibit any change in current in the presence of such a gas. Consequently, the two aforementioned example optical sensors would output two different optical signatures to the same gas due to the particular optical absorbance profile of that gas.

The optical signature detected by optical sensor 150 may also depend on the emission profile of radiation incident thereon.

FIG. 5B shows an optical absorbance profile 151A corresponding to a first gas, and an optical absorbance profile 151B corresponding to a second gas. FIG. 5B also shows a photosensitivity profile 151C for an example optical sensor 150.

In the FIG. 5B example, the wavelengths of radiation emitted are restricted to wavelengths in the range defined by arrow 151D. Because the emission profile of radiation overlaps optical absorbance profile 151A of the first gas and not optical absorbance profile 151B of the second gas, the optical signature detected by optical sensor 150 will only depend on the first gas.

Returning to FIG. 2A, in some embodiments, method 100 comprises obtaining training information (e.g., additional data 116) from sensing modalities instead of or in addition to photoelectrochemical sensing (first information 112) and optical sensing (second information 114). In some embodiments, the sensing modalities used in method 100 to obtain additional data 116 may include (but are in no way limited to):
electrochemical sensing (e.g., causing a chemical reaction through diffusion of a fluid inside an electrolyte, the reaction producing a measurable electrical signal);
catalytic-based sensing (e.g., detecting heat change from fluid combustion on a catalytic bed);
photoionization detection (e.g., ionizing fluids with UV light and measuring the resulting current);
colourimetric sensing (e.g., reacting fluid with a reagent, causing a detectible colour change); and/or
the like.

In some embodiments, step 106 comprises additionally obtaining measurements of one or more of: a temperature of the training fluid, and a relative humidity of the training fluid. Such measurements may be incorporated into additional data 116.

Step 108 comprises adding an element 12A to the training data set 12.

FIG. 2B is a diagram of one element 12A of training data set 12 according to an example embodiment. Element 12A comprises the known composition 15 of the current training fluid 14 (from step 102), and the training information from training sensors having different sensing modalities (from step 106, respectively shown as first information 112 and second information 114). In some embodiments, element 12A also comprises additional data 116 (which may include temperature and/or relative humidity measurement(s) of training fluid 14 or sensed information about training fluid from other sensing modalities as described above). Such additional data 116 may also be acquired as part of step 106. In some embodiments, additional data 116 of training data element 12A may also comprise an emission profile from the one or more radiation emitters 130 used in connection with the photoelectrochemical sensors 140 used to obtain first information 112 and/or the optical sensors 150 used to obtain second information 114. Such emission profiles may be measured or may be known in association with the control of the radiation emitters 130.

As described herein, the term emission profile in relation to a radiation emitter 130 refers to one or more characteristics regarding a radiation emitter 130. As an example, an emission profile may include:
the electrical operating conditions of radiation emitter 130;
a wavelength or range of wavelengths emitted by radiation emitter 130; and/or
the like.

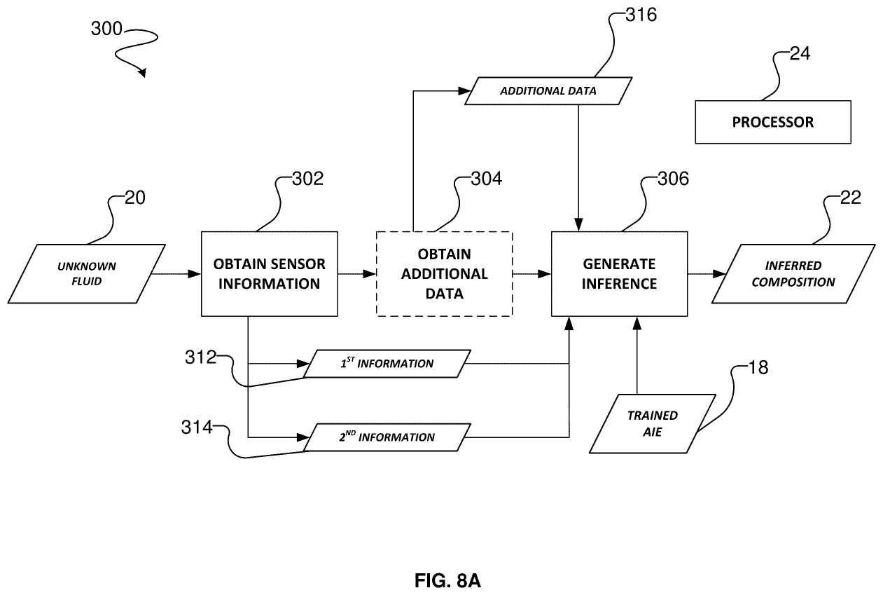

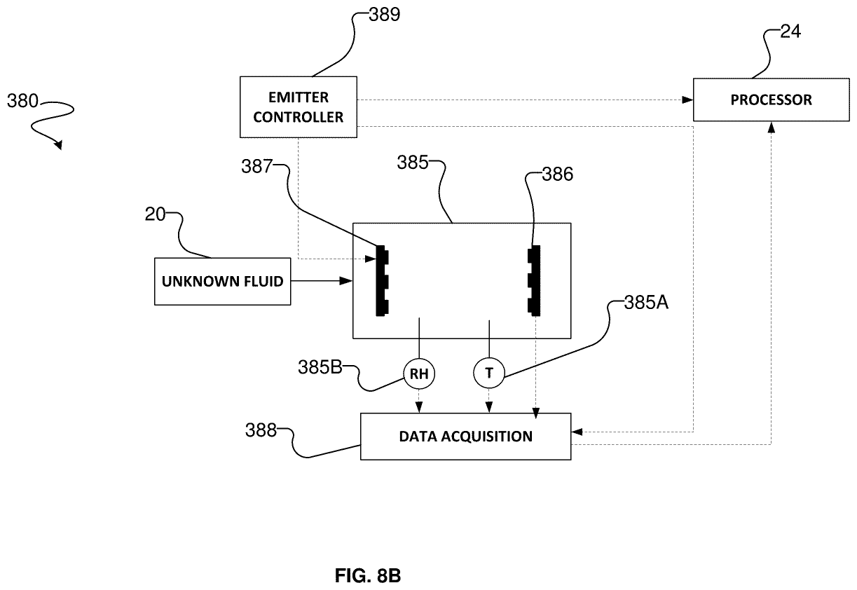

Step 109 comprises assessing whether to repeat steps 102 to 108 (i.e., whether to conduct another trial 101) to generate an additional element 12A in training data set 12. Steps 102 to 108 are repeated a plurality of times to provide a large number of trials 101 and a correspondingly large number of data elements 12A in training data set 12. By way of non-limiting example, the step 109 decision of whether to conduct another trial 101 could be based on:
whether the number of trials 101 already conducted exceeds a configurable threshold;
whether there is enough diversity in training data set 12 to render further tests unnecessary (e.g., if the range of training fluid compositions that have been trialed cover the expected compositions of unknown fluid 20 that are expected to be encountered by method 300 in FIG. 8A and/or apparatus 380 in FIG. 8B); and/or
the like.

Step 110 optionally comprises purging the test chamber of the training fluid 14 between successive trials 101 to avoid cross contamination of the training fluid between trials. The test chamber may be purged using an inert gas (for example, nitrogen).

In some embodiments, in each successive trial 101, a different parameter is changed at step 111 relative to the previous trial 101. Each trial 101 could involve changing, relative to the previous trial 101, one or more of the following parameters in the test chamber:
A known composition 15 of training fluid 14 (e.g., concentration of a particular compound in the training fluid 14 could be increased or decreased, or a new compound could be added to training fluid 14).
An emission profile of the one or more radiation emitters 130, for example:
   A radiation intensity of one or more of the radiation emitters 130 (e.g., one or more of the radiation emitters 130 could be driven at progressively increasing or decreasing currents over the course of several successive trials 101).
   An emission timing of the one or more radiation emitters 130 (e.g., the one or more radiation emitters 130 could each be activated sequentially or concurrently during the course of one trial 101).
   An emission duration of the one or more radiation emitters 130 (e.g., the one or more radiation emitters 130 could be pulsed, or driven for a configurable time interval during the course of a trial 101).
   A wavelength or range of wavelengths of radiation emitted by the one or more radiation emitters 130.
A temperature of the training fluid 14.
A relative humidity of the training fluid 14.
A characteristic of one or more of the one or more sensors (e.g., the one or more sensors on sensing array 186 in sensing chamber 185 (FIG. 6A)). For example, in the case of the photoelectrochemical sensors 140:
   Photoelectrochemical sensors 140 made using different synthesis techniques could be substituted. For example, a photoelectrochemical sensor 140 that is prepared using PVD with a direction of evaporation that is normal to the substrate surface could be substituted with a photoelectrochemical sensor 140 that is prepared using PVD with a direction of evaporation that is at a 75 degree angle to the substrate surface.
   An un-doped photoelectrochemical sensor 140 could be substituted for another photoelectrochemical sensor 140 doped with gold.

By way of example, steps 102 to 108 (trials 101) could be repeated for progressively increasing concentrations of a particular compound in the training fluid 14 at a fixed temperature, a fixed relative humidity, and a fixed emission profile.

As an example, training fluids 14 for a particular set of trials 101 could comprise a mixture of carbon dioxide, nitrogen, and oxygen. Over successive trials 101, the concentration of carbon dioxide in corresponding training fluids 14 could be increased from 0.1 ppm to 5 ppm in increments of 0.1 ppm.

As discussed above, the range of compositions of training fluids 14 that are sampled during each trial 101 could be based on expected ranges of the composition of unknown fluid 20 in step 300 (FIG. 1) and/or method 300 and/or apparatus 380 (see FIG. 8A and FIG. 8B respectively). By way of example, preparing a training data set 12 that will be used to obtain a trained AIE 18 for use in inferring a composition of flue gas in a flue gas outlet (as unknown fluid 20 of FIGS. 1, 8A, and 8B) may comprise conducting trials 101 of training fluids 14 with high carbon dioxide concentrations. By contrast, preparing a training data set 12 that will be used to obtain a trained AIE 18 for use in inferring a composition of air in a domestic setting, such as a house (unknown fluid 20 of FIGS. 1, 8A, and 8B) may comprise conducting trials 101 of training fluids 14 with comparatively low carbon dioxide concentrations.

Returning to FIG. 1, after generating training data set 12 in step 100, method 10 proceeds to step 200 which comprises training an AIE on training data set 12.

FIG. 6A is an apparatus 180 for generating training data according to an example embodiment. In some embodiments, training data generation method 100 (FIG. 2A) is conducted using apparatus 180.

Apparatus 180 comprises a plurality of fluids (e.g., gases) 182, which in the example embodiment of FIG. 6A comprises air 182A, nitrogen 182B, carbon dioxide 182C, and methane 182D, but which in general may comprise any fluids.

In apparatus 180 of the FIG. 6A embodiment, a composition of training fluid 14 is controlled by composition controller 181. Composition controller 181 controls control valve 183A, control valve 183B, control valve 183C, and control valve 183D, each of which respectively controls air 182A, nitrogen 182B, carbon dioxide 182C, and methane 182D. Actuating (opening and closing) control valves 183 controls a composition of training fluid 14.

Apparatus 180 optionally includes a mixer 184 that mixes training fluid 14 to promote even mixing of the constituents of training fluid 14.

After optional mixer 184, training fluid 14 is delivered to test chamber 185. Apparatus 180 comprises one or more photoelectrochemical sensors 140 and one or more optical sensors 150. Apparatus 180 also comprises one or more radiation emitters 130. Radiation emitters 130 are located and/or otherwise configured (e.g., using suitable optical components) to direct radiation onto photoelectrochemical sensors 140 and optical sensors 150.

In some embodiments, test chamber 185 comprises sensor array 186 comprising one or more photoelectrochemical sensors 140 and one or more optical sensors 150. In some embodiments, test chamber 185 comprises radiation emitter array 187 comprising one or more radiation emitters 130 configured to direct radiation onto sensor array 186.

Sensor array 186 comprises sensors comprising at least two sensing modalities (e.g., one or more photoelectrochemical sensors 140 and one or more optical sensors 150). Suitable data acquisition electronics (not shown in FIG. 6A) may be connected to acquire sensed information (e.g., first information 112, second information 114, additional data 116) from sensor array 186.

FIG. 6E is a diagram of a sensor array 186 according to an example embodiment. In some embodiments, sensors comprising different sensing modalities are present on sensor array 186. For example, sensor array 186 may comprise one or more photoelectrochemical sensors 140 and one or more optical sensors 150.

FIG. 6F is a diagram of an emitter array 187 according to an example embodiment. As shown in FIG. 6F, emitter array 187 comprises a plurality of radiation emitters 130.

Referring back to FIG. 6A, an emission profile of one or more radiation emitters 187A on emitter array 187 is controlled by emitter controller 189.

Data acquisition module 188 receives information (e.g., first information 112 and second information 114 and, in some cases, additional data 116) from the one or more training sensors in sensor array 186. Data acquisition module 188 may also receive an emission profile of radiation emitters 187 from emitter controller 189 and/or apparatus 180 may comprise a suitable sensor (not shown) for measuring the emission profile. Such emission profile information may be provided as part of additional data 116. In some embodiments, apparatus 180 comprises a temperature sensor 185A and data acquisition module 188 receives a corresponding temperature measurement of training fluid 14 from temperature sensor 185A. Such temperature information may be provided as part of additional data 116. In some embodiments, apparatus 180 comprises a humidity sensor 185B and data acquisition module receives a corresponding relative humidity measurement of training fluid 14 from relative humidity sensor 185B. Such humidity information may be provided as part of additional data 116.

Data acquisition module 188 also receives information from composition controller 181 regarding the known composition 15 of training fluid 14.

Data acquisition module 188 may associate first information 112, second information 114, and additional data 116 with a known composition 15 of the training fluid 14 in an element 12A of training data set 12.

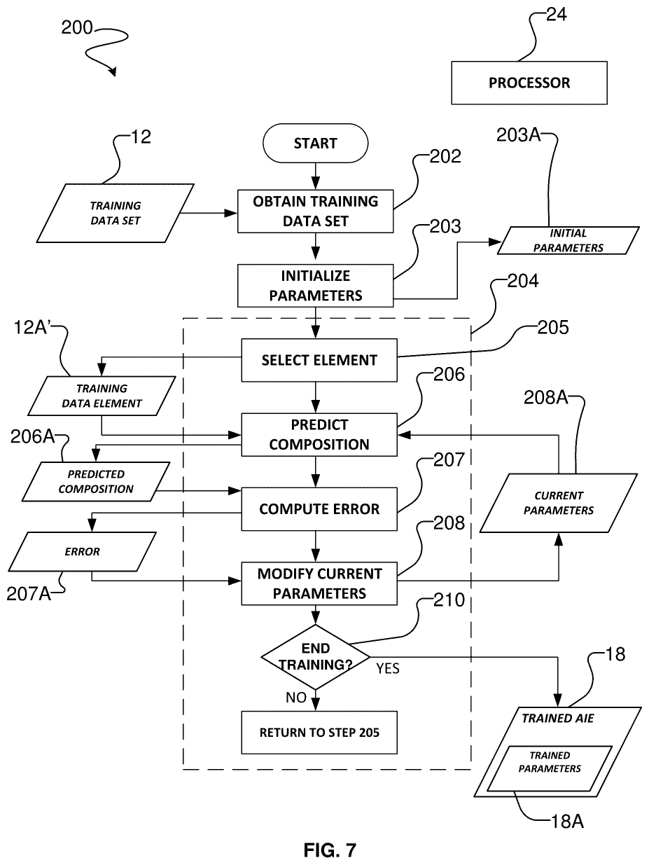

FIG. 7 is a flow chart of a method 200 for training an AIE to obtain a trained AIE 18 comprising a plurality of trainable parameters 18A according to an example embodiment.

Method 200 commences in block 202 which comprises obtaining training data set 12. In some embodiments, training data set 12 is obtained using method 100 and/or apparatus 180.

Step 203 comprises initializing the trainable parameters of the AIE to obtain initial trainable parameters 203A.

Block 204 comprises performing a plurality of training iterations to ultimately obtain a trained AIE 18 comprising trained parameters 18A. Each iteration of block 204 involves modifying a current set of trainable AIE parameters 208A to obtain a new set of current AIE parameters 208A. In the first iteration of block 204, initial trainable parameters 203A provide the current set of trainable parameters 208A input into block 204. In subsequent iterations, the modified set of current of trainable parameters 208A output from the previous iteration of block 204 provide the current set of trainable parameters 208A input into block 204. In the last iteration of block 204, the modified set of current parameters 208A are output as trained parameters 18A of AIE 18.

Block 204 commences with step 205 which comprises selecting (e.g. sampling, randomly sampling, selectively procuring) an element 12A′ of training data set 12 (see FIG. 2B).

Step 206 comprises, for the selected element 12A′, predicting a composition 206A of the training fluid using the AIE based at least in part on current trainable parameters 208A, first information 112, second information 114, and (optionally) additional information 116 (see FIGS. 2A, 2B).

Step 207 comprises computing an error 207A between the step 206 predicted composition 206A and the known composition 15 associated with the selected element 12A′ (see FIG. 2A and FIG. 2B).

In some embodiments, determining the step 207 error comprises computing a value of a loss function based on the known composition 15 of the selected element 12A′, and predicted composition 206A.

Step 208 comprises modifying the current parameters 208A of the AIE based on the block 207 error 207A. In some embodiments, step 208 comprises modifying current parameters 208A in an effort to reduce error 207A between predicted composition 206A and the known composition 15 of the selected element 12A′.

Step 208 may comprise any suitable known machine learning technique to modify current trainable parameters 208A—e.g. back propagation, similar machine learning techniques, other machine learning techniques and/or the like. For example, step 208 could comprise taking partial derivatives of the block 207 loss (e.g. loss function) 207A with respect to each of the trainable AIE parameters to ascertain relative amounts of change to make to the respective current parameters 208A.

Step 210 comprises evaluating a training conclusion condition. If one or more training conclusion conditions are fulfilled, then method 200 is finished and outputs trained AIE 18 comprising current trainable parameters 208A as modified in the last iteration of block 208. Otherwise, method 200 comprises returning to step 205 to complete another iteration of block 204.

In some embodiments, the one or more block 210 training conclusion conditions comprise:
whether the block 207 error 207A drops below a configurable threshold for a configurable threshold number of iterations;
whether the block 207 error 207A has not reduced by more than a configurable threshold amount over a configurable threshold number of training iterations;
whether the number of training iterations has exceeded a configurable threshold;
user input indicating that training has concluded; and/or
the like.

FIG. 8A is a flowchart of a method 300 for inferring a composition 22 of an unknown fluid 20 according to an example embodiment. Method 300 may be performed by processor 24 which may be embodied by the same processor(s) used for other methods described herein and/or by different processor(s). FIG. 8B is a schematic of an apparatus 380 for inferring a composition 22 of an unknown fluid 20 according to an example embodiment. In some embodiments, method 300 is conducted using apparatus 380.

Apparatus 380 is similar in many respects to apparatus 180 described above in connection with FIG. 6A. In particular, apparatus 380 comprises test chamber 385, sensor array 386 (including photoelectrochemical sensor 140 and optical sensor 150), emitter array 387, optional temperature sensor 385A, optional humidity sensor 385B, data acquisition module 388 and emitter controller 389 which may be respectively analogous to test chamber 185, sensor array 186 (including photoelectrochemical sensor 140 and optical sensor 150), emitter array 187, optional temperature sensor 185A, optional humidity sensor 185B, data acquisition unit 188 and emitter controller 189 of apparatus 180 described above in connection with FIG. 6A. In some embodiments, inference method 300 (FIG. 8A) and training data generation method 100 (FIG. 2A) are implemented by the same apparatus—e.g., apparatus 180 and 380 are the same physical apparatus.

Method 300 commences in step 302 which comprises obtaining sensor information (first information 312 and second information 314) about unknown fluid 20 from sensors having two different sensing modalities. In some embodiments, step 302 comprises obtaining first information 312 from one or more photoelectrochemical sensors 140, and obtaining second information 314 from one or more optical sensors 150 which may form part of sensor array 386 and which may be illuminated with radiation from emitter array 387 which may comprise one or more radiation emitters 130. First information 312 and second information 314 may be obtained from sensors 140 and 150 using data acquisition module 388 which may include suitable signal processing electronics (not expressly shown). In some such embodiments, first information 312 associated with one or more photoelectrochemical sensors 140 may take the form of one or more photoelectrochemical profiles as described above with respect to first information 112, and second information 314 from one or more optical sensors 150 may take the form of one or more optical signatures as described above with respect to second information 114.

In some embodiments, sensor information (e.g. first information 112 and second information 114) is obtained from photoelectrochemical sensors 140 and optical sensors 150 that are identical to the photoelectrochemical sensors 140 and optical sensors 150 of apparatus 180.

Method 300 then proceeds to optional step 304 which comprises obtaining additional data 316 about unknown fluid 20. In some embodiments, additional data 316 comprises an emission profile of the one or more radiation emitters 130 on emitter array 387 which may be controlled by emitter controller 389 and/or processor 24. In some embodiments, additional data 316 comprises a temperature measurement of unknown fluid 20 from temperature sensor 385A which may be obtained via data acquisition module 388. In some embodiments, additional data 316 comprises a relative humidity measurement for unknown fluid 20 from relative humidity sensor 385B which may be obtained via data acquisition module 388.

Step 306 comprises inferring a composition (inferred composition 22) of unknown fluid 20. The step 306 inference of inferred composition 22 may comprise using first information 312, second information 314, and trained AIE 18 to infer a composition of unknown fluid 20. The step 306 inference of inferred composition 22 may additionally be based on optional additional data 316.

Method 300 is particularly advantageous in that it can leverage sensor information from a plurality of sensors with different sensing modalities at the same time to obtain an inference regarding the composition of unknown fluid 20 with improved accuracy relative to using each sensing modality on its own and/or relative to using the sensing modalities independently.

By way of example, it might not be possible to assess accurately a composition of unknown fluid 20 using just one sensing modality in isolation due to there being a plurality of constituents in unknown fluid 20. By acquiring sensing information (e.g., first information 312 and second information 314) about unknown fluid 20 from sensors having two different sensing modalities, and by generating an inference using a trained AIE 18 that is trained on training data 12 covering expected compositions of the particular unknown fluid 20, a more accurate inference regarding the composition of unknown fluid 20 can be made relative to using each sensing modality on its own and/or relative to using the sensing modalities independently

Method 300 has utility, inter alia, at industrial facilities. In industrial facilities, there are typically one or more legacy sensors for sensing gas compositions. By way of example, method 300 could be used on a flue gas outlet to infer a composition of flue gas. In such a case, the flue gas outlet could already have a pre-existing carbon dioxide sensor.

Such legacy sensors (or other legacy sensors) can be used to further refine (e.g. fine tune) trained AIE 18.

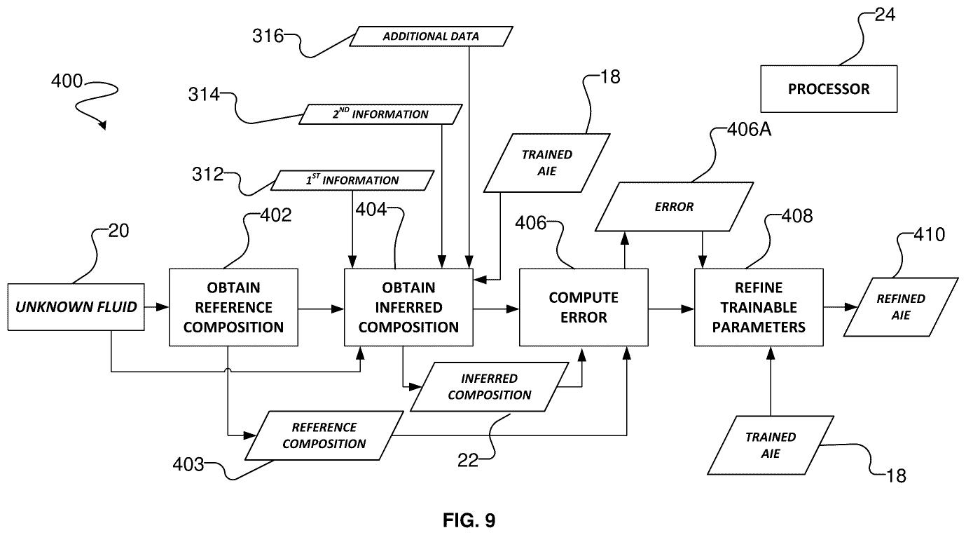

FIG. 9 is a flow chart of a method 400 for obtaining a refined AIE 410 according to an example embodiment. Method 400 may be performed by processor 24 which may be embodied by the same processor(s) used for other methods described herein and/or by different processor(s).

Method 400 commences at step 402 which comprises obtaining a reference composition 403 for unknown fluid 20. In some embodiments, reference composition 403 is obtained from a legacy sensor (not expressly shown in FIG. 9) at an industrial facility, as described above, or in any other suitable location. In some embodiments, the reference composition is a known historical value of a particular constituent in unknown fluid 20. For example, in the context of an industrial facility, the carbon dioxide content of flue gas from a boiler might have a known average value for a particular boiler output.

Step 404 comprises inferring a composition (inferred composition 22) of unknown fluid 20 using method 300.

Step 406 comprises computing an error between reference composition 403 and inferred composition 22.

In some embodiments, determining the step 406 error 406A comprises computing a value of a loss function based on reference composition 403 and inferred composition 22.

Step 408 comprises refining the trainable parameters of trained AIE 18 (or refining additional trained parameters which can be added to trained AIE 18) based on the step 406 error 406A to thereby obtain refined AIE 410. In some embodiments, step 408 comprises modifying the trainable parameters of trained AIE 18 (or modifying additional trained parameters which can be added to trained AIE 18) to reduce error 406A between inferred composition 22 and reference composition 403 to thereby obtain refined AIE 410. Step 408 could comprise any suitable known machine learning technique to modify the trainable parameters—e.g., back propagation, similar machine learning techniques, other machine learning techniques and/or the like. For example, any of the machine learning techniques described above in connection with FIG. 7 may be used in step 408. Method 400 could be iterated as many times as there are available reference compositions 403 to further refine refined AIE 410.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### Interpretation of Terms

Where a component (e.g. an optical sensor, a photoelectrochemical sensor, an emitter, a test chamber, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the present technology.

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Software and other modules may reside on servers, workstations, personal computers, tablet computers, image data encoders, image data decoders, PDAs, color-grading tools, video projectors, audio-visual receivers, displays (such as televisions), digital cinema projectors, media players, and other devices suitable for the purposes described herein. Those skilled in the relevant art will appreciate that aspects of the system can be practised with other communications, data processing, or computer system configurations, including: Internet appliances, hand-held devices (including personal digital assistants (PDAs)), wearable computers, all manner of cellular or mobile phones, multi-processor systems, microprocessor-based or programmable consumer electronics (e.g., video projectors, audio-visual receivers, displays, such as televisions, and the like), set-top boxes, color-grading tools, network PCs, mini-computers, mainframe computers, and the like.

The invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. A method for preparing a training data set for training an artificial intelligence engine (AIE) operable to infer a composition of a fluid, the method comprising:
conducting a plurality of trials to generate a plurality of elements of the training data set, each trial comprising:
obtaining a training fluid with a known composition;
delivering the training fluid to a test chamber;
obtaining first training information about the training fluid in the test chamber using one or more first training sensors having a first sensing modality;
obtaining second training information about the training fluid in the test chamber using one or more second training sensors having a second sensing modality, the second sensing modality different from the first sensing modality; and
associating, in a computer-interpretable format, the known composition with the first training information and the second training information to provide an element of the training data set.

2. The method as defined in claim 1 wherein the first sensing modality comprises photoelectrochemical sensing, the one or more first training sensors comprise one or more photoelectrochemical sensors each comprising semiconductor material in electrical contact with one or more electrodes, each photoelectrochemical sensor operable to sense a change in electrical characteristics at the one or more electrodes when the semiconductor material interacts with the training fluid in the presence of incident radiation, and wherein the first training information is based on an output from the one or more photoelectrochemical sensors.

3. The method as defined in claim 2 wherein the one or more photoelectrochemical sensors comprise a plurality of photoelectrochemical sensors and the plurality of photoelectrochemical sensors comprises one or more of:
each of the plurality of sensors comprising different crystalline structures;
each of the plurality of sensors doped with different dopants;
each of the plurality of sensors comprising different metallic nanoparticles comprising one or more of: Pt, Au, Ru, Pd, Ru, Rh, Ir, one or more oxides thereof, one or more complexes thereof, or a combination thereof; and
each of the plurality of sensors fabricated using different synthesis techniques.

4. The method as defined in claim 2 wherein the output from each of the one or more photoelectrochemical sensors comprises one or more photoelectrochemical response profiles, each photoelectrochemical response profile comprising a time dependent electrical characteristic measured at the one or more corresponding electrodes.

5. The method as defined in claim 4 wherein, for each trial, obtaining the first training information about the training fluid comprises one or more of:
calculating a rate of change of the photoelectrochemical response profile and including the rate of change in the first training information; and
calculating a maximum value of the photoelectrochemical response profile and including the rate of change in the first training information.

6. The method as defined in claim 1 wherein the second sensing modality comprises optical sensing, the one or more second training sensors comprise optical sensors, and wherein the second training information is based on an output of the one or more optical sensors.

7. The method as defined in claim 6 wherein the output of the one or more optical sensors comprises one or more electrical signals representative of an optical signature of the training fluid.

8. The method as defined in claim 1 wherein:
the first sensing modality comprises photoelectrochemical sensing, the one or more first training sensors comprise one or more photoelectrochemical sensors each comprising semiconductor material in electrical contact with one or more electrodes, each photoelectrochemical sensor operable to sense a change in electrical characteristics at the one or more electrodes when the semiconductor material interacts with the training fluid in the presence of incident radiation, and wherein the first training information is based on an output from the one or more photoelectrochemical sensors;
the second sensing modality comprises optical sensing, the one or more second training sensors comprise optical sensors, and wherein the second training information is based on an output of the one or more optical sensors;
wherein the method comprises, for each trial:
emitting radiation from at least one radiation emitter, the radiation directed to impinge on the one or more first training sensors and the one or more second training sensors; and
associating, in the computer-interpretable format, an emission profile of the at least one radiation emitter with the element of the training data set.

9. The method as defined in claim 8 comprising varying the wavelengths of radiation emitted from the at least one radiation emitter between successive trials.

10. The method as defined in claim 1 comprising:
for each trial, one or both of:
obtaining a humidity measurement of the training fluid, and associating, in the computer-interpretable format, the humidity measurement with the element of the training data set; and
obtaining a temperature measurement of the training fluid, and associating, in the computer-interpretable format, the temperature measurement with the element of the training data set.

11. A method for training an artificial intelligence engine (AIE) operable to infer a composition of a fluid, the method comprising:
(a) employing the method of claim 1 to generate a training data set;
(b) providing an AIE comprising trainable parameters;
(c) initializing values for the trainable parameters;
(d) performing a plurality of training iterations to obtain a trained AIE comprising trained parameters, each training iteration comprising, for an element of the training data set:
(i) predicting a composition of the training fluid based at least in part on the first training information, the second training information, and current values of the trainable parameters;
(ii) determining an error between the predicted composition of the training fluid from step (i) and the known composition of the training fluid; and
(iii) modifying, based at least in part on the error calculated in step (ii), the current values of the trainable parameters to obtain updated parameters.

12. The method as defined in claim 11 wherein the element of the training data set comprises additional data comprising one or more of:
an emission profile;
a relative humidity measurement of the training fluid; and
a temperature measurement of the training fluid; and
step (i) comprises predicting a composition of the training fluid for the at least one element of the training data set based at least in part on the first training information, the second training information, the current values of the trainable parameters, and the additional data.

13. A method of inferring a composition of a fluid, the method comprising:
providing one or more first deployed sensors having the first sensing modality, and one or more second deployed sensors having the second modality;
acquiring the trained AIE from claim 11;
obtaining first information about the fluid from the one or more first deployed sensors;
obtaining second information about the fluid from the one or more second deployed sensors; and
inferring, using the trained AIE, an inferred composition of the fluid based at least in part on the first information, the second information, and the trained parameters of the trained AIE.

14. The method as defined in claim 13 comprising:
obtaining additional deployed data about the fluid, the additional deployed data comprising one or more of:
a deployed emission profile of one or more deployed radiation emitters emitting radiation directed to impinge on the fluid;
a deployed temperature measurement of the fluid; and
a deployed relative humidity measurement of the fluid; and
inferring, using the AIE, the inferred composition of the fluid based at least in part on the first information, the second information, the trained parameters of the AIE, and the additional deployed data.

15. The method as defined in claim 13 wherein:
the first deployed sensors comprise the first training sensors; and
the second deployed sensors comprise the second training sensors.

16. The method as defined in claim 13 comprising:
obtaining a reference composition of the fluid;
determining a reference error between the inferred composition of the fluid and the reference composition of the fluid; and
modifying the values of the trained parameters based on the reference error.

17. The method as defined in claim 16 comprising obtaining the reference composition from one or more of:
a legacy sensor operable to determine the reference composition; and
historical expected values for the composition of the fluid.

18. A method of inferring a composition of a fluid, the method comprising:
conducting a plurality of trials to generate a plurality of elements of a training data set, each trial comprising:
obtaining a training fluid with a known composition;
delivering the training fluid to a test chamber;
obtaining first training information about the training fluid in the test chamber using one or more first training sensors having a first sensing modality;
obtaining second training information about the training fluid in the test chamber using one or more second training sensors having a second sensing modality, the second sensing modality different from the first sensing modality;
associating, in a computer-interpretable format, the known composition with the first training information and the second training information to provide an element of the training data set;
providing an AIE comprising trainable parameters;
initializing values for the trainable parameters;
performing a plurality of training iterations to obtain a trained AIE comprising trained parameters, each training iteration comprising for an element of the training data set:
(i) predicting a composition of the training fluid based at least in part on the first training information, the second training information, and current values of the trainable parameters;
(ii) determining an error between the predicted composition of the training fluid from step (i) and the known composition of the training fluid; and
(iii) modifying, based at least in part on the error calculated in step (ii), the current values of the trainable parameters to obtain updated parameters;
providing one or more first deployed sensors having the first sensing modality, and one or more second deployed sensors having the second modality;
acquiring the trained AIE;
obtaining first information about the fluid from the one or more first deployed sensors;
obtaining second information about the fluid from the one or more second deployed sensors; and
inferring, using the trained AIE, an inferred composition of the fluid based at least in part on the first information, the second information, and the trained parameters of the trained AIE.

19. An apparatus for determining the composition of fluids, the apparatus comprising:
one or more first sensors having a first sensing modality;
one or more second sensors having a second sensing modality; and
a processor configured to:
obtain first information about an unknown fluid from the one or more first sensors;
obtain second information about the unknown fluid from the one or more second sensors; and
infer, using a trained AIE comprising trained parameters, an inferred composition of the unknown fluid based at least in part on the first information, the second information, and the trained parameters of the trained AIE.

20. The apparatus of claim 19 wherein:
the one or more first sensors comprise one or more photoelectrochemical sensors each comprising semiconductor material in electrical contact with one or more electrodes, each photoelectrochemical sensor operable to sense a change in electrical characteristics at the one or more electrodes when the semiconductor material interacts with the unknown fluid in the presence of incident radiation, and wherein the first information is based on an output from the one or more photoelectrochemical sensors; and
the one or more second sensors comprise one or more optical sensors, and wherein the second information is based on an output of the one or more optical sensors.
